(19) United States
(12) Patent Application Publication
(10) Pub. No.: US 2025/0123199 A1
(43) Pub. Date: Apr. 17, 2025

(54) PROCESS FOR OPERATING AN OPTICAL GAS MEASURING SYSTEM FOR DETERMINING THE CONCENTRATION OF MEASURING GAS, WHICH HAS A LINE SPECTRUM, OPTICAL GAS MEASURING SYSTEM AND COMPUTER PROGRAM PRODUCT

(71) Applicant: Dräger Safety AG & Co. KGaA, Lübeck (DE)

(72) Inventor: Oliver WILLE, Lübeck (DE)

(21) Appl. No.: 18/906,314

(22) Filed: Oct. 4, 2024

(30) Foreign Application Priority Data
Oct. 11, 2023 (DE) 10 2023 127 745.6

(51) Int. Cl. G01N 21/31; G01N 21/03; G01N 33/00

(52) U.S. Cl. CPC G01N 21/31; G01N 21/03; G01N 33/0027

(57) ABSTRACT

A process for operating an optical gas measurement system determines a concentration of measuring gas, which has a line spectrum, and includes steps for determining an indication of ambient pressure. A corresponding optical gas measurement system determines a concentration of measuring gas, which has a line spectrum, and determines an indication of ambient pressure. A computer program product includes instructions that cause the optical gas measurement system to perform the process steps.

### CROSS REFERENCE TO RELATED APPLICATIONS

This application claims the benefit of priority under 35 U.S.C. § 119 of German Application 10 2023 127 745.6, filed Oct. 11, 2023, the entire contents of which are incorporated herein by reference.

### TECHNICAL FIELD

The present invention pertains to a process for operating an optical gas measuring system for determining the concentration of measuring gas which has a line spectrum, a corresponding optical gas measuring system and a corresponding computer program product.

### BACKGROUND

Optical gas measurement systems and processes for operating optical gas measurement systems are known. Optical gas measuring systems have a sample chamber which can be filled with a measuring gas to be measured, also referred to as target gas, or with a sample gas comprising the measuring gas. Certain properties of the measuring gas or the sample gas, in particular its absorption properties, can be measured by suitable detectors, making it possible to determine the concentration of the measuring gas in the sample chamber.

Such optical gas measuring systems, hereinafter also referred to simply as gas measuring systems, are known, for example, from DE 197 13 928 C1, DE 196 11 290 A1 and EP 3 504 535 B1.

The concentration determination is usually based on signals generated by at least one measurement detector and at least one reference detector. A quotient is usually formed from the signals of these detectors in order to compensate for an error acting simultaneously on both detectors. However, some influences that affect both detectors simultaneously cannot be compensated for in this way.

It is known that the density of the measuring gas or the sample gas in the sample chamber is influenced by the ambient pressure in accordance with the ideal gas law. At low ambient pressure, the density is low, i.e. there are fewer gas molecules in the sample chamber, so that fewer molecules of the measuring gas are present, even though the concentration of the measuring gas in the sample chamber has not changed. However, due to the reduced number of gas molecules in the sample chamber, the absorption properties of the gas in the sample chamber change, so that a correspondingly calculated concentration of the measuring gas is incorrectly lower than the actual concentration. At high ambient pressure, the density of the measuring gas or sample gas is higher and the corresponding calculated concentration of the measuring gas is incorrectly higher than it actually is. If the ambient pressure is increased from 1000 mbar to 1200 mbar, for example, the calculated concentration without compensation for the influence of pressure (pressure compensation) is 20% higher than the actual concentration.

In order to at least partially compensate for the falsification of the calculated concentration of the measuring gas, it is therefore known to provide a pressure sensor which generates a corresponding pressure signal and to use the pressure signal to correct the calculated concentration of the measuring gas, for example taking Lambert-Beer's law into account.

A gas measuring device with pressure compensation is known, for example, from EP 3 370 057 A1.

However, such known solutions require the provision of a pressure sensor, which increases the cost of manufacturing the gas measuring device. With corrosive measuring gases, there is also the problem that corrosion and thus failure of the pressure sensor can occur. The selection of a corrosion-resistant pressure sensor further increases the cost of the gas measuring device.

### SUMMARY

It is an object of the invention to provide a process for operating an optical gas measuring system for determining the concentration of measuring gas which has a line spectrum, a corresponding optical gas measuring system and a corresponding computer program product which enable the ambient pressure to be estimated (provides an indicator of ambient pressure) without a pressure sensor having to be provided.

These and other objects are attained according to the invention.

This disclosure including the description, the figures and the claims provide features and advantageous embodiments of the invention.

According to the invention, a process is thus provided for operating an optical gas measuring system for determining the concentration of measuring gas by radiation absorption, the measuring gas having a line spectrum.

The process comprises the steps of a) receiving a first measurement signal which indicates a first radiation intensity at a first measurement wavelength or at a first measurement wavelength range, b) receiving a second measurement signal which indicates a second radiation intensity at a second measurement wavelength or at a second measurement wavelength range, wherein the first measurement wavelength or the first measurement wavelength range is different from the second measurement wavelength or from the second measurement wavelength range, c) calculating a determination value which indicates a quotient of the received first measurement signal and the received second measurement signal, d) receiving comparison information indicating a plurality of possible quotients of the first measurement signal and the second measurement signal for a corresponding plurality of possible ambient pressures, e) comparing the determination value with the comparison information to determine a quotient of the plurality of possible quotients, which has the highest correspondence with the determination value, f) determining which ambient pressure of the possible ambient pressures the quotient of the plurality of possible quotients corresponds to in order to obtain an estimated ambient pressure (an indication of the current ambient pressure), and g) providing the estimated ambient pressure.

In the context of the invention, it was recognized that the so-called effect of pressure broadening of measuring gases with a line spectrum can be used to determine and provide information about the ambient pressure acting on the measuring gas.

Measuring gases with a line spectrum are, for example, methane and carbon dioxide. When the pressure of a measuring gas with a line spectrum changes, in addition to the effect of the change in density described above, there is also a change in the line spectrum, namely a broadening when the pressure increases and a narrowing when the pressure decreases. This effect is referred to as pressure broadening. The invention is based on the idea of using the pressure-dependent change in the line spectrum to draw conclusions about the ambient pressure. This is achieved by receiving measurement signals of two different measurement wavelengths or measurement wavelength ranges, i.e. information is obtained at two different points or regions in the spectrum. Both measurement signals are related to each other and thus form a characteristic value, namely a quotient, which is therefore dependent on the current ambient pressure. By comparing the quotient with corresponding predetermined comparison information, it is then possible to determine which of the possible ambient pressures the quotient corresponds to and consequently the estimated ambient pressure can be determined and provided.

In the simplest embodiment of the invention, the estimated ambient pressure is merely provided and can be used in various ways. For example, it is possible to output the estimated ambient pressure to a user of the gas measurement system for informational purposes. Furthermore, if a pressure sensor is provided for determining the ambient pressure, it is possible to enable the estimated ambient pressure to be determined redundantly to this sensor, which can improve the reliability of the determination of the ambient pressure. It is also possible to use the information about the estimated ambient pressure for at least partial pressure compensation. Further uses of the estimated ambient pressure are possible.

In the context of the invention, a measuring gas is understood to be a gas or a gas mixture whose concentration in a gas or gas mixture to be examined (the measuring gas) can be determined by means of absorption spectroscopy and which also has a line spectrum.

A measurement signal is a signal that indicates a radiation intensity at a measurement wavelength or at a measurement wavelength range. A measurement signal is available from a radiation detector.

Measuring wavelength ranges that differ from each other are understood to be measuring wavelength ranges that differ from each other by at least one wavelength. For example, measuring wavelength ranges are different from each other if their center wavelengths are different.

A determination value is understood to be a value that indicates a quotient of the received first measurement signal and the received second measurement signal. In one example, the determination value is obtained by directly forming a quotient from the received first measurement signal and the received second measurement signal. In a further example, the determination value is obtained by forming a quotient from the concentration signals of two gas measuring devices which are not identical in construction, the respective concentration signal indicating a quotient of the measurement signal and the reference signal.

Comparison information is understood to be information that indicates a number of possible quotients from the first measurement signal and the second measurement signal for a corresponding number of possible ambient pressures. The comparison information can be available in essentially any way and can be provided, for example, as a look-up table. In a further example, the comparison information can be obtained by executing a predetermined calculation rule. The comparison information may be dependent on further parameters.

Preferably, step c) of the process is performed at a first determination time to obtain a first determination value and (again) performed at a later second determination time to obtain a second determination value, wherein the process additionally comprises the step of: d1) determining a difference between the second determination value and the first determination value, and wherein step e) of the process is performed when the difference between the second determination value and the first determination value exceeds a predetermined threshold value.

In this way, the robustness of the process can be increased by determining the estimated ambient pressure only if there is a change in the determination value that exceeds the predetermined threshold value.

Preferably, step c) is carried out in a continuously repeating manner so that first determination values and second determination values are obtained continuously.

Preferably, the process further comprises the step of: b1) receiving a reference signal indicating a third radiation intensity at a reference wavelength or at a reference wavelength range, wherein the reference wavelength is different from the first measuring wavelength and/or the second measuring wavelength or wherein the reference wavelength range is different from the first measuring wavelength range and/or the second measuring wavelength range.

By shifting the reference wavelength or the reference wavelength range into a range that is different from at least one measuring wavelength or at least one measuring wavelength range, preferably from both measuring wavelengths or both measuring wavelength ranges, the influence of the pressure broadening on the measuring gas concentration in the range of low measuring gas concentrations can be advantageously reduced.

Preferably, the process further comprises the steps of: b2) determining a concentration of the measuring gas from the first measurement signal and/or from the second measurement signal and from the reference signal, h) at least partially correcting the determined concentration based on the estimated ambient pressure to obtain a corrected concentration of the measuring gas, and i) providing the corrected concentration of the measuring gas.

In this way, the determined concentration of the measuring gas can be at least partially corrected, taking into account the estimated ambient pressure.

The concentration of the measuring gas can be determined from the measurement signal(s) and the reference signal in a known manner.

Preferably, the measuring gas comprises methane and/or carbon dioxide.

In this way, it is possible to realize the invention in known gas measuring devices by simply adding a further suitable measuring signal to the two measuring signals or reference signals that are usually already present.

In the case of carbon dioxide, there is also the advantage that carbon dioxide is present in almost every environment on earth, so that in this case a gas measuring device can be provided to provide the estimated ambient pressure, in which each additional radiation detector is free to measure other gases. This would work comparably well with methane, but methane is not present in almost every environment.

According to the invention, an optical gas measuring system for determining the concentration of measuring gas, which has a line spectrum, by radiation absorption, is also provided.

The optical gas measurement system comprises: a first radiation detector arranged to indicate a first radiation intensity at a first measurement wavelength or at a first measurement wavelength range, a second radiation detector arranged to indicate a second radiation intensity at a second measurement wavelength or at a second measurement wavelength range, and an evaluation unit configured to perform some or all of the steps of the process described above.

A radiation detector is understood to be a component that is set up to measure electromagnetic radiation, namely light. A radiation detector can be configured, for example, as a photoresistor, a photodiode, a phototransistor, a bolometer, a pyroelectric detector, a thermoelectric detector and/or a thermal detector. Further examples of suitable configurations of radiation detectors are detector arrays, line detectors, CCD sensors and CMOS sensors.

Each embodiment of a radiation detector can have one or more optical filters. Each embodiment of a radiation detector can additionally or alternatively have one or more digital filters. By means of the optical and/or digital filter, hereinafter also referred to generically as a filter, the respective radiation detector can be set up to indicate the respective radiation intensity.

An optical filter is a component of the gas measuring system that selects the incident radiation according to predetermined criteria. Such a filter can, for example, be configured as a bandpass filter or double bandpass filter and select the radiation according to a wavelength or one or more wavelength ranges. The optical filter can, for example, be configured as a filter disk or as a tunable filter.

Each radiation detector, i.e. the first radiation detector and/or the second radiation detector and/or each further radiation detector can be configured as an independent component or as part of a multiple detector, for example as part of a double detector or as part of a quadruple detector. The radiation detectors can be of the same or different configuration.

It is possible that several radiation detectors are neither independent nor part of a multiple detector, but are provided by a common radiation detector, which provides a corresponding number of signals by switching between a number of filters and thus simultaneously provides several radiation detectors within the meaning of the invention. For example, the common radiation detector can provide the first measurement signal and the second measurement signal by switching between a first filter and a second filter. The same applies to each further measurement signal and reference signal, which can also be provided by the one radiation detector or another common radiation detector by means of suitable filters.

Each of the first, second and further filters can be present as a physical component and can be arranged in the beam path in front of the common radiation detector and/or in front of another common radiation detector or can be configured as a digital filter.

In a non-limiting example, the first radiation detector and the second radiation detector can be configured as components of a common component, for example a double detector or a quadruple detector. In addition to the first radiation detector and the second radiation detector, further radiation detectors may be present, such as a third and a fourth radiation detector. If the process comprises step b1), it is preferred that the gas measurement system

comprises a third radiation detector which is arranged to indicate the third radiation intensity at the reference wavelength or at the reference wavelength range.

It is generally preferred that the gas measurement system also has a fourth radiation detector, which is configured to indicate the fourth radiation intensity at a further reference wavelength or at a further reference wavelength range. The reference wavelength and the further reference wavelength or the reference wavelength range and the further reference wavelength range can be identical or different from each other.

Preferably, the first and third radiation detectors are provided in a first gas measuring device and the second and fourth radiation detectors are provided in a second gas measuring device. This can be done by providing one or more radiation detectors and/or by providing one or more common radiation detectors.

The gas measurement system according to the invention can have further elements, such as a sample chamber for holding the sample gas, one or more components influencing the radiation propagation (mirrors, reflectors, lenses and the like), one or more radiation sources, a control unit which can be configured to form the evaluation unit and/or one or more energy storage devices for providing energy for operating the gas measurement system.

The one or more radiation sources can be set up to emit light comprising ultraviolet radiation and/or infrared radiation. In this respect, ultraviolet radiation is understood to mean electromagnetic radiation with a wavelength of 10 nm to 400 nm, preferably from 100 nm to 380 nm. In this respect, infrared radiation is understood to mean electromagnetic radiation with a wavelength of 0.750 μm to 1000 μm. The one or more radiation sources can emit the light in broadband and/or narrowband. For example, the one or more radiation sources can be configured as one or more LEDs and/or as one or more incandescent lamps.

The evaluation unit can be configured as a hardware and/or software component for carrying out the process according to the invention and preferably also for controlling the components of the gas measurement system. For example, the evaluation unit can be configured as a software module of a computer or as a hardware and/or software module of a gas measuring device, which can be part of the gas measuring system, and can be, for example, a processor or a microcontroller or one or more processors with associated memory unit(s).

The energy storage device(s) can be configured as an accumulator and/or a battery, for example.

The gas measuring system can be formed by a portable, mobile or stationary gas measuring device with an open or closed cuvette or can be configured as a distributed system, which can comprise one or more gas measuring devices.

The gas measuring system can be of an open-path configuration, whereby one system component can have the radiation source or sources and another system component can have the first and second radiation detectors. In a further variant, the other system component has the first radiation detector and another system component has the second radiation detector.

Preferably, the optical gas measuring system has a cuvette which defines an absorption path, whereby the length of the absorption path is at least 10 cm, preferably at least 80 cm.

The provision of such an absorption section has proven to be particularly favorable with regard to the measuring properties of the gas measuring system.

In accordance with the invention, there is further provided a computer program product, the computer program product comprising instructions that cause the above-described optical gas measurement system to perform some or all of the above-described process steps. The computer program product may include a computer readable storage medium (or media) having computer readable program instructions thereon for causing a processor to carry out aspects of the present invention. The computer readable storage medium can be a tangible device and can retain and store instructions that are non-transitory (for example a program/code stored with the memory unit otherwise accessible for execution) for use by an instruction execution device such as the evaluation unit or the control unit.

Any disclosure relating to the process is also deemed to be disclosed in connection with the gas measuring system and vice versa. Any disclosure relating to the gas measurement system is also deemed to be disclosed in connection with the computer program and vice versa. Any disclosure relating to the process is also deemed to be disclosed in connection with the computer program and vice versa.

These and other features, effects and advantageous embodiments of the invention are also apparent from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a flow diagram showing an example of a process according to the invention;

FIG. 2 is a schematic graph representation of the effect of pressure broadening in a pressure-concentration diagram;

FIG. 3 is a schematic graph representation of possible quotients of the measurement signals depending on concentration and pressure;

FIG. 4 is a schematic view of an embodiment of a gas measurement system according to the invention; and

FIG. 5 is a schematic view of another example of a gas measurement system according to the invention.

### DESCRIPTION OF PREFERRED EMBODIMENTS

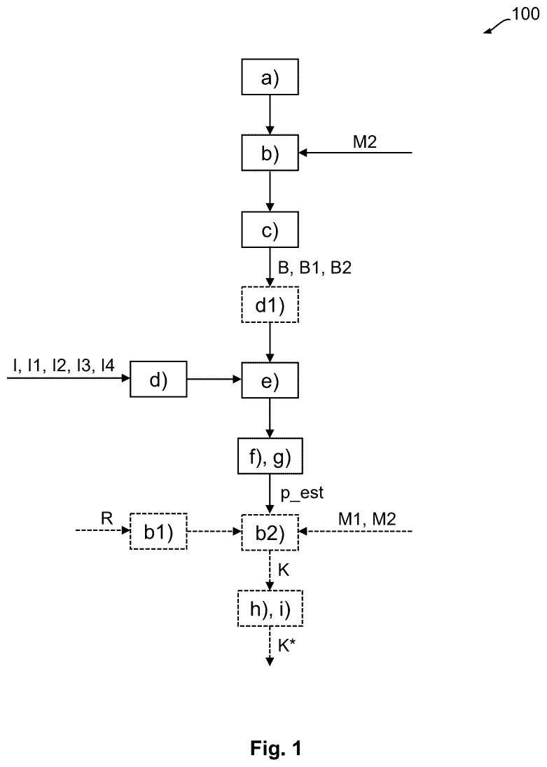

Referring to the drawings, according to the invention, a process 100 is provided for operating an optical gas measurement system 200 for determining the concentration of measuring gas by radiation absorption, wherein the measuring gas has a line spectrum. An embodiment example of such a process 100 is shown in FIG. 1. The embodiment example according to FIG. 1 also has optional steps shown as dashed lines.

The process 100 comprises at least steps a), b), c), d), e), f) and g).

Step a) is the reception of a first measurement signal M1, which indicates a first radiation intensity at a first measurement wavelength or at a first measurement wavelength range.

Step b) is the reception of a second measurement signal M2, which indicates a second radiation intensity at a second measurement wavelength or at a second measurement wavelength range.

The first measuring wavelength or the first measuring wavelength range is different from the second measuring wavelength or from the second measuring wavelength range.

Step c) is the calculation of a determination value B, B1, B2, which indicates a quotient Q of the received first measurement signal M1 and the received second measurement signal M2.

Step d) is the reception of comparison information I, I0, I1, I2, I3, which indicates a plurality of possible quotients from the first measurement signal M1 and the second measurement signal M2 for a corresponding plurality of possible ambient pressures p0, p1.

Step e) is to compare the determination value B, B1, B2 with the comparison information I, I1, I2, I3 to determine a quotient I1 of the plurality of possible quotients I, I0, I1, I2, I3 which has the highest agreement with the determination value B, B1, B2.

Step f) is to determine with which ambient pressure p of the possible ambient pressures p0, p1 the quotient I1 of the plurality of possible quotients I, I0, I1, I2, I3 corresponds in order to obtain an estimated ambient pressure p_est.

Step g) is the provision of the estimated ambient pressure p_est.

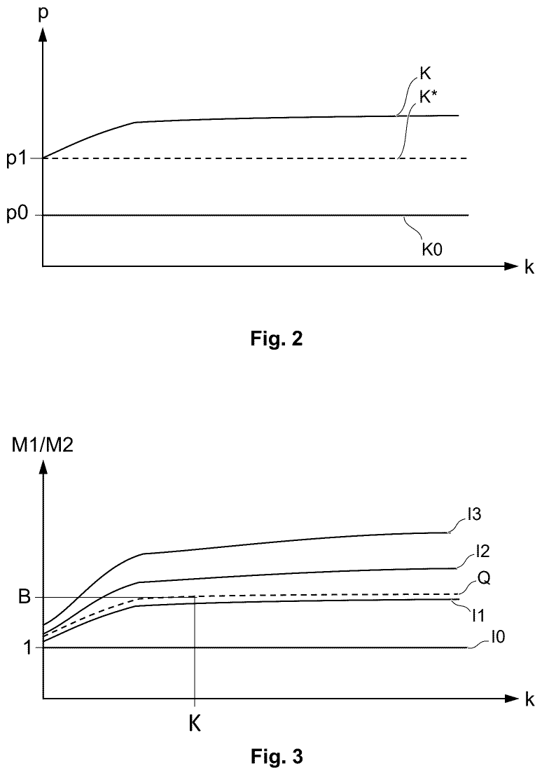

As explained above, the invention is based on the idea of using the pressure-dependent change in the line spectrum to draw conclusions about the ambient pressure. The effect of pressure broadening in a gas measuring device without pressure compensation is shown schematically in FIG. 2 in a pressure-concentration diagram, in which the ambient pressure p is plotted against the concentration k of the measuring gas. If the concentration k of the measuring gas increases without a change in pressure, a gas measuring device at a calibration pressure p0 (applicable) displays the concentration K0 as the concentration. However, if a pressure different from the calibration pressure p0 is present, the effect of pressure broadening causes the determined concentration K to deviate from the actual concentration K*. However, by knowing the ambient pressure p1, proving an at least partially compensated determined concentration K* is possible.

The mode of operation of steps c), d), e) and f) of the invention is explained in more detail in connection with FIG. 3.

In FIG. 3, possible quotients 10, I1, I2, I3 of the measurement signals M1, M2 depending on the concentration k are schematically plotted as a set of curves for corresponding ambient pressures p0, p1, p2, p3, which are known from the comparison information. In step c) of the process, a determination value B is calculated, which indicates a quotient Q of the received first measurement signal M1 and the received second measurement signal M2 as a function of the concentration k, for example at a currently present concentration K, of the measurement gas. In step e), the determination value B is compared, for example, with the set of curves of possible quotients 10, I1, I2, I3 in order to determine the quotient I1 which has the highest agreement with the determination value B. From the comparison information, it is known which pressure p1 corresponds to the quotient I1 determined in this way, so that this assignment can be used to determine what the estimated ambient pressure p_est is.

It is also possible to determine a corresponding estimated ambient pressure p_est by interpolation between two curves of the set of curves, in the example between I1 and 12, which takes into account the deviation from one of the existing curves, if any.

In a variant not shown, the comparison information is not formed by a predetermined set of curves, but is obtained by executing a calculation rule, for example using an empirically determined function which assigns the determination value to a corresponding estimated ambient pressure.

As shown in FIG. 1, the process 100 may further optionally comprise that step c) is performed at a first determination time to obtain a first determination value B1, and that step c) is performed at a later second determination time to obtain a second determination value B2, wherein the process 100 may additionally comprise the step: d1) determining a difference between the second determination value B2 and the first determination value B1, wherein step e) is performed in this case when the difference between the second determination value B2 and the first determination value B1 exceeds a predetermined threshold value.

Step c) can optionally be carried out in a continuously repeating manner, so that first determination values B1 and second determination values B2 are obtained continuously. As shown in FIG. 1, the process 100 may further optionally comprise step b1).

Step b1) is the reception of a reference signal R which indicates a third radiation intensity at a reference wavelength or at a reference wavelength range, wherein the reference wavelength is different from the first measurement wavelength and/or from the second measurement wavelength or wherein the reference wavelength range is different from the first measurement wavelength range and/or from the second measurement wavelength range.

As shown in FIG. 1, the process 100 may further optionally comprise steps b2), h) and i).

Step b2) is the determination of a concentration K of the measuring gas from the first measurement signal M1 and/or from the second measurement signal M2 and from the reference signal R.

Step h) is the at least partial correction of the determined concentration K based on the estimated ambient pressure p_est to obtain a corrected concentration K* of the measuring gas.

Step i) is to provide the corrected concentration K* of the measuring gas.

The measuring gas can comprise methane and/or carbon dioxide.

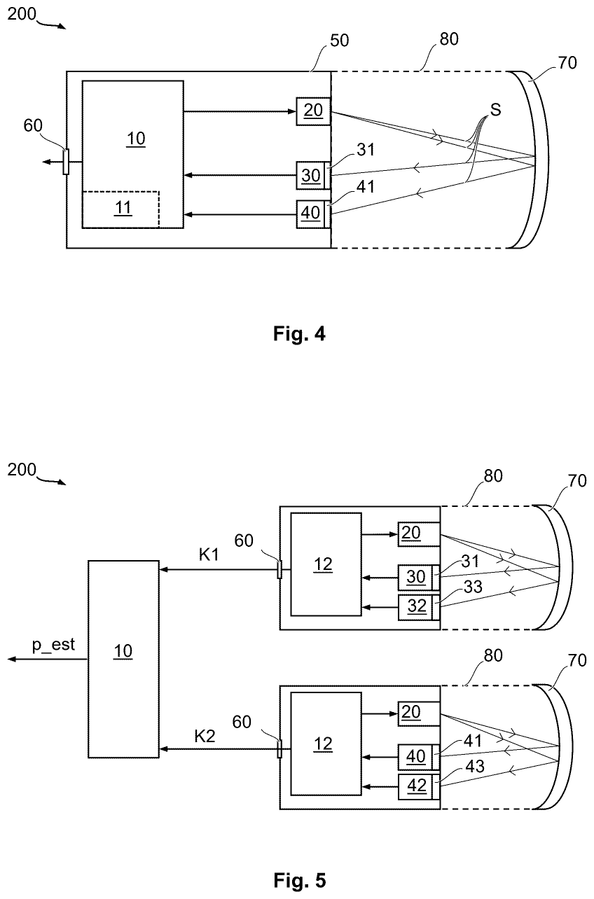

According to the invention, a gas measuring system 200 for determining the concentration of measuring gas, which has a line spectrum, is also provided by radiation absorption. Examples of embodiments of gas measuring systems 200 according to the invention are shown in FIG. 4 and FIG. 5.

Each gas measuring system 200 according to the invention comprises a first radiation detector 30, which is set up to indicate a first radiation intensity at a first measuring wavelength or at a first measuring wavelength range and to which a first optical filter 31 is preferably assigned, a second radiation detector 40 which is set up to indicate a second radiation intensity at a second measurement wavelength or at a second measurement wavelength range and to which a second optical filter 41 is preferably assigned, and an evaluation unit 10, which is set up to carry out some or all of steps a), b), . . . of the process 100.

The gas measurement system 200 can also have further components, for example a memory unit 11, which can be configured as part of the evaluation unit 10, a housing 50 for accommodating some or all of the components of the gas measurement system 200, a reflector 70 and a data interface 60 for providing and/or outputting information, such as the estimated ambient pressure p_est and/or the determined concentration K and/or the corrected concentration K*.

The gas measurement system 200 can have further and/or other components than those shown in FIGS. 4 and 5.

In FIG. 4, the gas measuring system 200 is configured as a gas measuring device, which has a reflector 70 arranged outside the housing 50, which reflects the radiation from the radiation source 20—shown schematically as radiation rays S—and directs the radiation in the direction of the first radiation detector 30 and second radiation detector 40. The radiation passes through the sample space formed between the housing 50 and the reflector 70 by a cuvette 80 and is (partially) absorbed by any measuring gas present.

However, the gas measurement system 200 can also be configured as a distributed system in which the radiation source 20 and the radiation detectors 30, 40 are arranged locally separated from each other in different components of the system and aligned with each other, i.e. in an open-path design which does not require a reflector 70. In this case, the cuvette is an open cuvette, i.e. not a structural component.

The gas measuring system 200 according to FIG. 5 is configured as a variant of a distributed system, which comprises two gas measuring devices according to FIG. 4 and a data processing unit 10, which forms the evaluation unit 10. However, unlike the gas measuring device according to FIG. 4, the first gas measuring device has the first radiation detector 30 and a third radiation detector 32, which indicates a third radiation intensity at a reference wavelength or at a reference wavelength range, and the second gas measuring device has the second radiation detector 40 and a fourth radiation detector 42, which indicates a fourth radiation intensity at a second reference wavelength or at a second reference wavelength range. The gas measuring devices of this gas measuring system 200 are set up to provide, via respective data interfaces 60, a concentration K1, K2 of the measuring gas calculated from the respective signals for the evaluation unit 10, which is set up according to the invention to determine the estimated ambient pressure p_est. The gas measuring devices of this gas measuring system 200 therefore furthermore, in contrast to the gas measuring device according to FIG. 4, do not each have an evaluation unit 10, but instead each have a control unit 12, which are set up to control the respective gas measuring device.

Not shown is a computer program product according to the invention, which is also provided and comprises instructions that cause the above-described optical gas measurement system 200 to perform some or all of the above-described steps a), b), . . . of the process 100. The computer program product may include a computer readable storage medium (or media) having computer readable program instructions thereon for causing a processor to carry out aspects of the present invention. The computer readable storage medium can be a tangible device that is non-transitory and can retain and store instructions that are non-transitory (for example a program/code stored with the memory unit 11 or otherwise accessible for execution) for use by an instruction execution device such as the evaluation unit 10 or the control unit 12.

While specific embodiments of the invention have been shown and described in detail to illustrate the application of the principles of the invention, it will be understood that the invention may be embodied otherwise without departing from such principles.

### LIST OF REFERENCE CHARACTERS

10 Evaluation unit
11 Memory unit
12 Control unit
20 Radiation source
30 First radiation detector
31 First optical filter
32 Third radiation detector
33 Third optical filter
40 Second radiation detector
41 Second optical filter
42 Fourth radiation detector
43 Fourth optical filter
50 Housing
60 Interface
70 Reflector
80 Cuvette
100 Process
200 Gas measuring system, optical gas measuring system
a), b), . . . Steps of the process
B Determination value
B1 First determination value
B2 Second determination value
k, K, K0, . . . Concentration of the measuring gas
K* Corrected concentration of the measuring gas
I, I0, I1, . . . Comparison information
M1 First measurement signal
M2 Second measurement signal
p, p0, p1 Environmental pressure
p_est Estimated ambient pressure (indicator of ambient pressure)
Q Quotient
R Reference signal
S Radiation (rays)

What is claimed is:

1. A process for operating an optical gas measuring system for determining a concentration of measuring gas, which has a line spectrum, by radiation absorption, the process comprising the steps of:
a) receiving a first measurement signal, which indicates a first radiation intensity at a first measurement wavelength or at a first measurement wavelength range;
b) receiving a second measurement signal, which indicates a second radiation intensity at a second measurement wavelength or at a second measurement wavelength range, wherein the first measuring wavelength or the first measuring wavelength range is different from the second measuring wavelength or from the second measuring wavelength range;
c) calculating a determination value, which indicates a quotient of the received first measurement signal and the received second measurement signal,
d) receiving comparison information, which indicates a plurality of possible quotients of the first measurement signal and the second measurement signal for a corresponding plurality of possible ambient pressures;
e) comparing the determination value with the comparison information to determine a quotient of the plurality of possible quotients which has the highest agreement with the determination value;
f) determining which ambient pressure of the plurality of possible ambient pressures the quotient of the plurality of possible quotients corresponds to obtain an estimated ambient pressure; and
g) providing the estimated ambient pressure.

2. A process according to claim 1,
wherein step c) is performed at a first determination time to obtain a first determination value, and
wherein step c) is carried out at a later second determination time to obtain a second determination value,
wherein the process additionally comprises the step of d1) determining a difference between the second determination value and the first determination value, and
wherein step e) is carried out when the difference between the second determination value and the first determination value exceeds a predetermined threshold value.

3. A process according to claim 2, wherein step c) is carried out continuously repetitively, so that first determination values and second determination values are obtained continuously.

4. A process according to claim 1, further comprising the step of b1) receiving a reference signal which indicates a third radiation intensity at a reference wavelength or at a reference wavelength range, wherein the reference wavelength is different from the first measuring wavelength and/or from the second measuring wavelength or wherein the reference wavelength range is different from the first measuring wavelength range and/or the second measuring wavelength range.

5. A process according to claim 4, further comprising the steps of:
b2) determining a concentration of the measuring gas from the first measurement signal and/or from the second measurement signal and from the reference signal;
h) at least partially correcting the determined concentration based on the estimated ambient pressure to obtain a corrected concentration of the measuring gas; and
i) providing the corrected concentration of the measuring gas.

6. A process according to claim 1, wherein the measuring gas comprises methane and/or carbon dioxide.

7. A process according to claim 1, further comprising providing a computer program product with a computer readable storage medium that is a tangible device comprising non-transitory instructions that cause the optical gas measurement system to perform one or more of the process steps.

8. An optical gas measuring system for determining the concentration of measuring gas, which has a line spectrum, by radiation absorption, the optical gas measuring system comprising:
a first radiation detector, which is configured to indicate a first radiation intensity at a first measuring wavelength or at a first measuring wavelength range;
a second radiation detector, which is configured to indicate a second radiation intensity at a second measurement wavelength or at a second measurement wavelength range; and
an evaluation unit which is configured to
receive a first measurement signal from the first radiation detector, which indicates the first radiation intensity at the first measurement wavelength or at the first measurement wavelength range;
receive a second measurement signal from the second radiation detector, which indicates the second radiation intensity at the second measurement wavelength or at the second measurement wavelength range, wherein the first measuring wavelength or the first measuring wavelength range is different from the second measuring wavelength or from the second measuring wavelength range;
calculate a determination value, which indicates a quotient of the received first measurement signal and the received second measurement signal;
receive comparison information, which indicates a plurality of possible quotients of the first measurement signal and the second measurement signal for a corresponding plurality of possible ambient pressures;
compare the determination value with the comparison information to determine a quotient of the plurality of possible quotients which has the highest agreement with the determination value;
determine which ambient pressure of the plurality of possible ambient pressures the quotient of the plurality of possible quotients corresponds to obtain an estimated ambient pressure; and
provide the estimated ambient pressure.

9. An optical gas measuring system according to claim 8, further comprising a cuvette which defines an absorption path, wherein a length of the absorption path is at least 10 cm.

10. An optical gas measuring system according to claim 8, wherein the evaluation unit is configured to:
calculate a first determination value at a first determination time and to calculate a second determination value at a later second determination time;
determine a difference between the second determination value and the first determination value to, and
comparing the determination value with the comparison information when the difference between the second determination value and the first determination value exceeds a predetermined threshold value.

11. An optical gas measuring system according to claim 10, wherein the evaluation unit is configured to calculate the determination value continuously repetitively, so that first determination values and second determination values are obtained continuously.

12. An optical gas measuring system according to claim 8, wherein the evaluation unit is configured to receive a reference signal which indicates a third radiation intensity at a reference wavelength or at a reference wavelength range, wherein the reference wavelength is different from the first measuring wavelength and/or from the second measuring wavelength or wherein the reference wavelength range is different from the first measuring wavelength range and/or the second measuring wavelength range.

13. An optical gas measuring system according to claim 12, wherein the evaluation unit is configured to:
determine a concentration of the measuring gas from the first measurement signal and/or from the second measurement signal and from the reference signal;
at least partially correct the determined concentration based on the estimated ambient pressure to obtain a corrected concentration of the measuring gas; and
provide the corrected concentration of the measuring gas.

14. An optical gas measuring system according to claim 8, wherein the measuring gas comprises methane and/or carbon dioxide.

15. An optical gas measuring system according to claim 8, further comprising a computer program product with a computer readable storage medium that is a tangible device comprising non-transitory instructions executable by the evaluation unit.

16. A computer program product with a computer readable storage medium that is a tangible device comprising non-transitory instructions that cause an optical gas measurement system to perform one or more of the process steps comprising:
a) receiving a first measurement signal, which indicates a first radiation intensity at a first measurement wavelength or at a first measurement wavelength range;
b) receiving a second measurement signal, which indicates a second radiation intensity at a second measurement wavelength or at a second measurement wavelength range, wherein the first measuring wavelength or the first measuring wavelength range is different from the second measuring wavelength or from the second measuring wavelength range;
c) calculating a determination value, which indicates a quotient of the received first measurement signal and the received second measurement signal,
d) receiving comparison information, which indicates a plurality of possible quotients of the first measurement signal and the second measurement signal for a corresponding plurality of possible ambient pressures;
e) comparing the determination value with the comparison information to determine a quotient of the plurality of possible quotients which has the highest agreement with the determination value;
f) determining which ambient pressure of the plurality of possible ambient pressures the quotient of the plurality of possible quotients corresponds to obtain an estimated ambient pressure; and
g) providing the estimated ambient pressure,
wherein the optical gas measurement system comprises:
a first radiation detector, which is configured to indicate the first radiation intensity at the first measuring wavelength or at the first measuring wavelength range;
a second radiation detector, which is configured to indicate the second radiation intensity at the second measurement wavelength or at the second measurement wavelength range; and
an evaluation unit configured to execute the instructions.
